(19) United States
(12) Patent Application Publication
(10) Pub. No.: US 2005/0043363 A1
(43) Pub. Date: Feb. 24, 2005

(54) Efficient microbial preparation of capravirine metabolites M4 and M5

(75) Inventors: Shanghui Hu, San Diego, CA (US); Carlos Alberto Martinez, Oceanside, CA (US); Junhua Tao, San Diego, CA (US); Daniel Rida Yazbeck, San Diego, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/922,136

(22) Filed: Aug. 18, 2004

Related U.S. Application Data
(60) Provisional application No. 60/496,635, filed on Aug. 19, 2003.

(51) Int. Cl. C12P 13/06; C07D 43/02; A61K 31/4439

(52) U.S. Cl. 514/341; 546/274.4; 435/116

(57) ABSTRACT

The present invention provides a method for producing metabolites of capravirine (2-carbamoyloxymethyl-5-(3,5-dichlorophenyl)thio-4-isopropyl-1-(4-pyridyl)methyl-1H-imidazole) via whole cell biotransformation using fungi and bacterial cells as oxygenation catalysts.

### CROSS-REFERENCE TO RELATED APPLICATIONS

This application claims the benefit of U.S. Provisional Application Ser. No. 60/496,635, filed Aug. 19, 2003, which is incorporated by reference in its entirety.

### BACKGROUND OF THE INVENTION

The present invention relates to the production of capravirine metabolites M4 and M5 by using microbial cell strains as oxygen transfer catalysts. The method can be used to selectively prepare sufficient quantities of M4 and M5 for use in various drug activity studies. These two metabolites have potent antiviral activity, while exhibiting less toxicity than capravirine itself.

Capravirine (CPV, also known as S-1153), which is also known as 2-carbamoyloxymethyl-5-(3,5-dichlorophenyl)thio-4-isopropyl-1-(4-pyridyl)methyl-1H-imidazole, is classified as a non-nucleoside reverse transcriptase inhibitor (NNRTI) and is a potent anti-HIV agent. Capravirine has demonstrated activity against HIV strains that are resistant to other antiviral agents. U.S. Pat. No. 5,910,506 describes capravirine and other imidazole derivatives that are useful as anti-HIV agents, while U.S. Pat. No. 6,083,958 describes, in part, anti-HIV compositions that contain such imidazole derivatives.

Two proposed metabolites of capravirine, M4 and M5, were structurally postulated as being hydroxylated metabolites of the capravirine isopropyl group (see Ohkawa, T. et al. Xenobiotica, 1998, 28, 877). The antiviral activity and relative toxicity of these metabolites has not previously been determined. Also, the two metabolites have to date not been prepared or characterized, due to difficulties in their synthesis. In particular, it is difficult to use human-liver derived in vitro systems (e.g., human liver homogenates also known as microsomes) (Pelkonen O, Maenpaa J, Taavitsainen P, Rautio A, Raunio H. Inhibition and induction of human cytochrome P450 (CYP) enzymes. Xenobiotica 28: 1203-1253, 1998) to prepare a sufficient quantity of metabolites for structural characterization, since in general they can only be used to generate nanogram (ng) to microgram (μg) amounts of materials. Human liver samples used for these studies are usually obtained from human donors, and not only the ethical implications of such methodology, but also the limited amounts in which microsomes are offered from commercial sources, pose a great limitation for their use on an industrial scale. Microbial models of mammalian metabolism have been reported in the literature as an inexpensive, renewable and simple alternative for the preparation of drug metabolites (R. V. Smith and J. P. Rosazza: Microbial models of mammalian metabolism. J. Pharm. Sci. 11,1737-1759).

Accordingly, a need exists for preparing capravirine metabolites M4 and M5 in sufficient quantities of scale in order to characterize their relative antiviral activity, associated toxicity and to elucidate their structure. The present invention describes the use of microbial cells to obtain sufficient amounts of metabolites M4 and M5 for such activity studies and structural characterization.

### SUMMARY OF THE INVENTION

The present invention is directed to a method for preparing a metabolite of 2-carbamoyloxymethyl-5-(3,5-dichlorophenyl)thio-4-isopropyl-1-(4-pyridyl)methyl-1H-imidazole from a cell strain, comprising reacting the cell strain with 2-carbamoyloxymethyl-5-(3,5-dichlorophenyl)thio-4-isopropyl-1-(4-pyridyl)methyl-1H-imidazole, and collecting the metabolite. The invention is further directed to the preparation of CPV metabolites from dioxygenated precursors.

Preferred metabolites produced via the invention include:

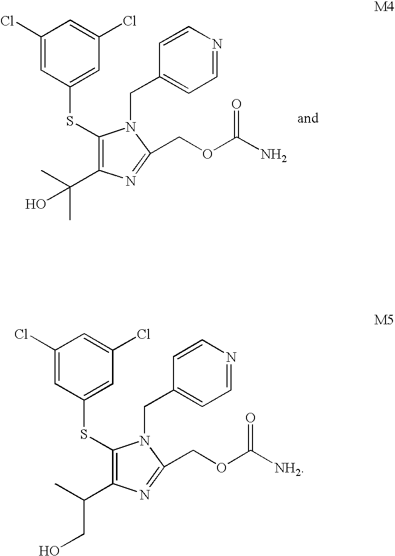

Preferred cell strains for use in the method include Streptomyces griseus ATCC 13273, Streptomyces griseolus ATCC 11796, Syncephalastrum racemosum ATCC 18192, Actinoplanes sp. ATCC 53771, Streptomyces rimosus ATCC 10970, Absidia pseudocylindrospora ATCC 24169, Mortierella isabellina ATCC 42613 and Verticillium theobromae ATCC 12474.

### DETAILED DESCRIPTION OF THE INVENTION

The preparation of metabolites M4 and M5 was achieved using microbial cell strains as oxygen transfer catalysts. Using this method, M4 and M5 can be produced at milligram to grams scale, and they can also be generated in a selective fashion. The methods described herein include a screening procedure, followed by a process optimization where fermentation parameters were optimized. In addition, a chemical method to convert undesired metabolites into M4 and M5 is also presented. Suitable bacterial and fungal strains were identified (see Table I and procedure below) from performing a microbial screening. Two particular bacterial strains, Streptomyces griseus ATCC 13273 and Streptomyces griseolus ATCC 11796, were found to be efficient in producing a mixture of M4 and M5 precursors which were chemically converted into M4 and M5. One fungal strain, Syncephalastrum racemosum ATCC 18192, selectively produced M4, which greatly facilitated the structural studies on this metabolite.

### DEFINITIONS

The term “ACN”, as used herein, refers to acetonitrile.

The term “CC₅₀”, as used herein, means the 50% cytotoxicity concentration, which is calculated as the concentration of compound that decreases the viability of uninfected, compound-treated cells to 50% of that of uninfected, compound-free cells.

The term “EC₅₀”, as used herein, means the statistically derived concentration of a toxicant that can be expected to cause a defined non-lethal effect in 50% of a given population of organisms under defined conditions.

The term “EC₉₀”, as used herein, means the statistically derived concentration of a toxicant that can be expected to cause a defined non-lethal effect in 90% of a given population of organisms under defined conditions.

The term “HPLC”, as used herein, refers to High Performance Liquid Chromatography, which is also often referred to as High Pressure Liquid Chromatography.

The term “MeOH”, as used herein, refers to methanol.

The term “min.”, as used herein, refers to minutes.

The term “NMR”, as used herein, refers to Nuclear Magnetic Resonance spectroscopy.

The term “RT”, as used herein, refers to room temperature.

The term “TFA”, as used herein, refers to trifluoroacetic acid.

The term “TLC”, as used herein, refers to Thin Layer Chromatography.

Experiments for Biosynthesis of CPV Metabolites M4 and M5

1. Microbial screening of CPV hydroxylators

Most of the microorganisms that were found to perform the desired reaction could also produce other dioxygenated metabolites such as M2 and M3, and in some cases dioxygenated compounds C12, C14 and M6 (SCHEME 1).

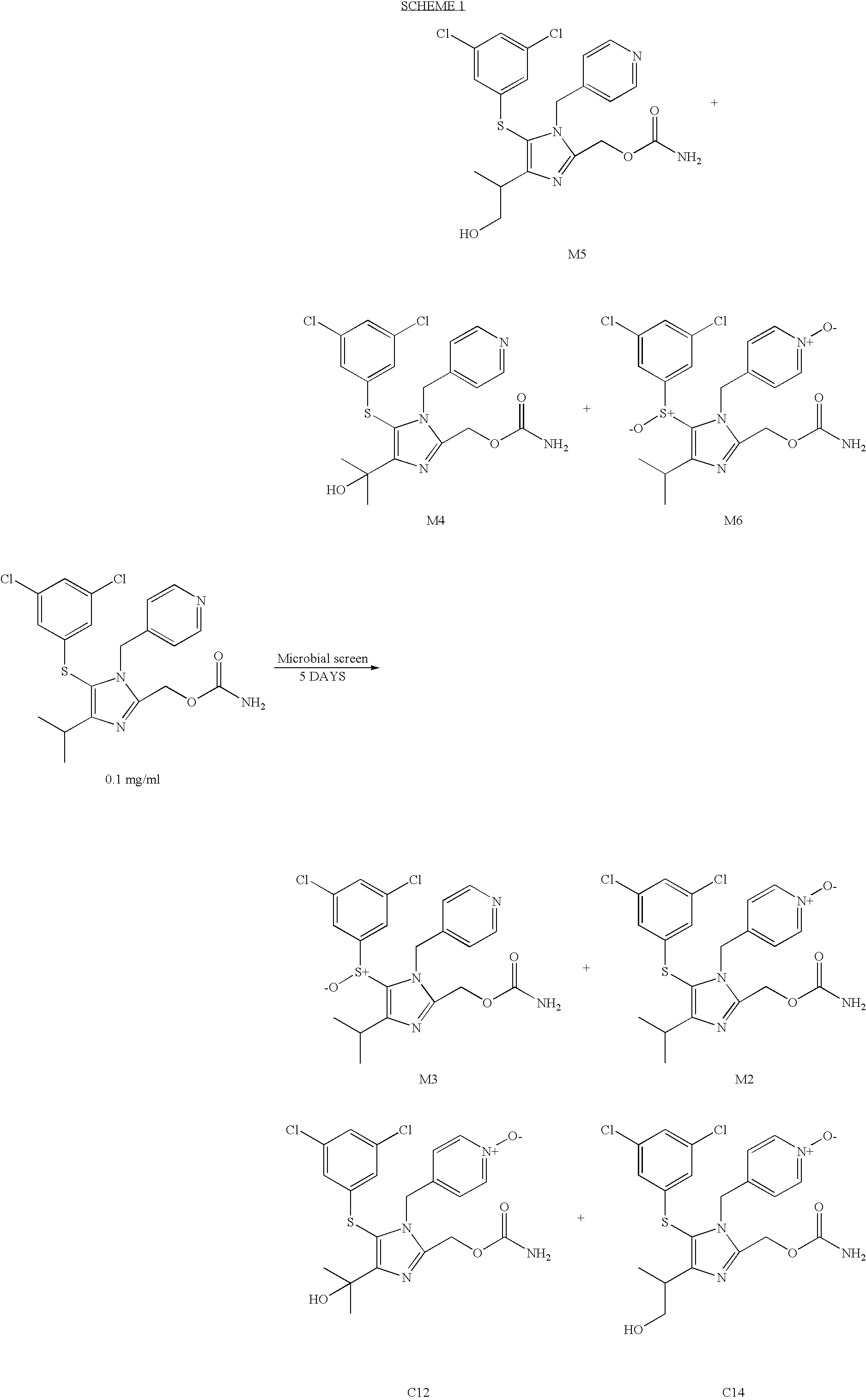

A brief description of the screening process as well as the reaction optimization and scale-up is summarized below. Twenty-eight different fungal strains and nineteen bacterial strains (see TABLE I) were grown from frozen stocks in agar plates. The plates comprised a mixture of 20 g glucose, 5 g soyflour, 5 g yeast extract, 5 g K₂HPO₄, 5 g NaCl, 1 g MgSO₄.7H₂O, 15 g bacto agar, completed with water to 1 L, adjusted pH to 7.2 and sterilized. Single colonies (bacteria) or a piece of the mycelia (fungi) were inoculated individually in 20 ml tubes containing 3 ml of the same growth media (no agar included). All strains were grown at 28° C. and 250 RPM on a rotary shaker. After 2 days, 0.3 mg of CPV was added from a 10% ethanol solution. After 5 days, the reactions were analyzed by HPLC using a Phenomenex Synergi Max RP C18 analyzed column with a flow rate of 1 ml/min and a gradient elution spanning from 5-95% acetonitrile and water (containing 0.1% TFA) to check for the presence of oxidation products.

TABLE I

| ATCC Number | Bacteria |
|---|---|
| 39647 | Acinetobacter sp. |
| 21536 | Bacillus sp. (lentus) |
| 49064 | Bacillus cereus |
| 11009 | Streptomyces argentoulus |
| 53771 | ctinoplanes sp. |
| 21271 | mycolatopsis mediterranel |
| 11796 | Streptomyces griseolus |
| 10137 | Streptomyces griseus sp. griseus |
| 13273 | Streptomyces griseus |
| 10970 | Streptomyces rimosus |
| 51531 | Chelatococcus asaccharovorans |
| 31338 | Nocardia corallina |
| 15592 | Rhodococcus sp. |
| 29347 | Pseudomonas oleovorans |
| 12633 | Pseudomonas putida |
| 17699 | Ralsonia eutropha |
| 21457 | Achromobacter lyticus Isono |
| 19795 | mycolatopsis orientalis |
| 35203 | Pseudocardia autotrophica |
| ATCC Number | Fungi |
| 34541 | Phanerochaete chiysosporium |
| 10404 | Rhizopus oryzae |
| 1008 | Aspergillus ochraceus |
| 18192 | Syncephalastrum racemosum |
| 9245 | Cunninghamella echinulata var. Elegans |
| 22751 | Absidia cylindrospora |
| 42613 | Mortierella isabellina |
| 18191 | Thamnidium elegans |
| 24169 | Absidia pseudocylindmspora |
| 10864 | Aspergillus niger |
| 9142 | Aspergillus niger |
| 15517 | Aspergillus parasiticus |
| 10029 | Aspergillus terreus |
| 7158 | Beauveria bassiana |
| 13144 | Beauveria bassiana |
| 10571 | Candida rugosa |
| 36190 | Cunninghamella echinulata |
| 13633 | Cutvularia lunata |
| 4740 | Mucor plumbeus |
| 36060 | Rhizopogon sp |
| 34541 | Cunninghamella echinulata var. Elegans |
| 12724 | Epicoccum oryzae |
| 12726 | Epicoccum sp. |
| 12725 | Epicoccum yuccae |
| 12722 | Epicoccum humicola |
| 16373 | Caldariomyces fumago |
| 28300 | Verticillium lecanii |
| 12474 | Verticillium theobromae |

Four bacterial strains were found to efficiently metabolize CPV under screening conditions: Actinoplanes sp. ATCC 53771, Streptomyces griseolus ATCC 11796, Streptomyces griseus ATCC 13273, and Streptomyces rimosus ATCC 10970. Of those, Streptomyces griseolus ATCC 11796 and Streptomyces griseus ATCC 13273 showed greater amounts of metabolites with almost complete consumption of starting material. M4 and M5, as well as the other metabolites shown in SCHEME 1, were observed during the screening process using these strains.

Four fungal strains were found to metabolize CPV under screening conditions: Absidia pseudocylindrospora ATCC 24169, Mortierella isabellina ATCC 42613, Verticillium theobromae ATCC 12474 and Syncephalastrum racemosum ATCC 18192. The fungus Syncephalastrum racemosum ATCC 18192 is preferred for selective conversion to the hydroxylated product M4. Other compounds present after the whole cell reaction included metabolites M2, M3 and unreacted CPV. Representative methods and reaction scale-up are shown for Syncephalastrum racemosum ATCC 18192 using conditions similar to the ones used in the screen.

2. Optimization studies using Streptomyces griseus ATCC 13273

Several experiments were conducted on bacterial strain Streptomyces griseus ATCC 13273 in order to optimize the entire cell reaction.

(a) Growth conditions

Glycerol-based media resulted in stable and high growth culture that ensured reproducibility of the procedure. A two-stage fermentation procedure was set up where preculture (first stage) was grown from fresh inoculum (colonies washed from agar plate) in shake flasks for 2 days. The second stage culture was started by adding preculture to fresh media (1/50-1/100 dilution) and the resulting culture was grown for 1 day before substrate was added from a 10% ethanol solution. SCHEME 2 below illustrates the conversion of CPV into metabolites C12, C14, M4, M5, and M6.

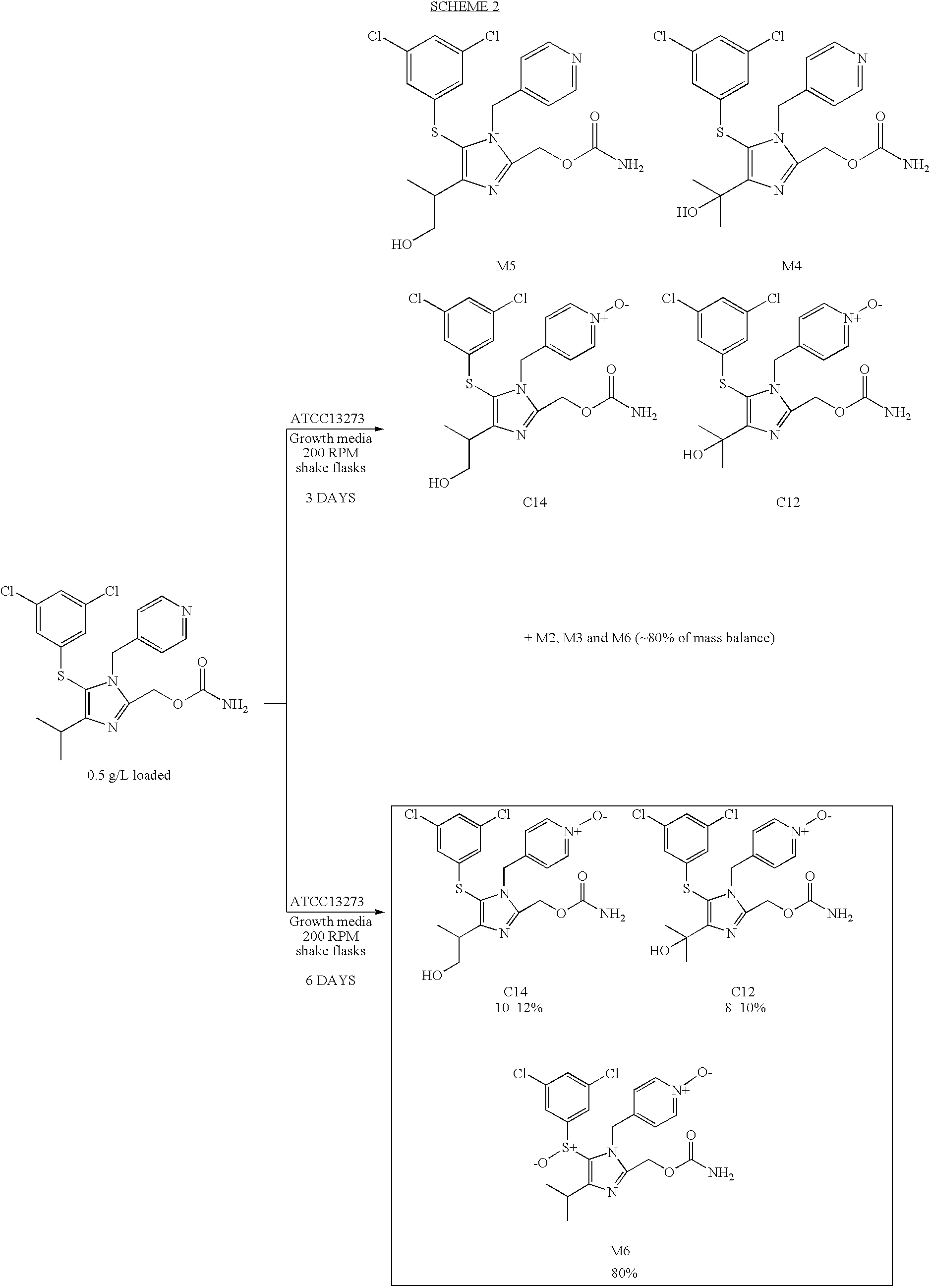

Close monitoring of reaction outputs indicated that the conversion of CPV into metabolites M4 and M5 peaked at about 3 days, followed by dioxygenation of those into C12 and C14, which continued until about 6 days (see SCHEME 2). The final crude materials after 6 days contained only three components: C12, C14 and M6.

(b) Development of HPLC method for the purification of C12 and C14

The crude extracts from the biotransformation of CPV were fractionated by semi-preparative chromatography on an Agilent HPLC preparative system. Multiple injections (extract dissolved in MeOH) loaded onto a 21.2×150 mm Phenomenex Max RP column (80 521 , 4 μm) were performed with UV detection at 254 nm and peak-level detection for fractionation adjusted to the injection volume. Gradient elution with a flow rate of 20 ml/min was used: 5% ACN/(0.1% TFA in water) for 2.9 min.; 5% to 15% in 0.1 min.; 15% to 45% in 12 min.; 45% to 98% in 1 min.; 98% for 4 min.; then reequilibration. Desired fractions were isolated and subsequent analysis by LC/MS and NMR showed that desired M4 and M5 fractions contained the corresponding N-oxide (C12 and C14 respectively). NMR and LC analysis further showed that the major component of each mixture was a higher oxidation product, C12 and C14. Therefore, these fractions were subjected to reduction conditions discussed below and were later repurified using the identical HPLC method to ensure >98% purity of product for clinical studies.

(c) Studies toward the N—O reduction of dioxygenated precursors C6:

Due to the small amount of pure M4 and M5 products and the presence of dioxygenated species C12 and C14, a model reaction for the reduction of M4 and M5 byproducts was devised (see SCHEME 3). Compound M6 was used as the test compound to study the reduction of the N-oxide moiety in these metabolites. The first method tested involved the use of diethylchlorophosphite (DECP). The reaction was unsuccessful and no further conditions were tested.

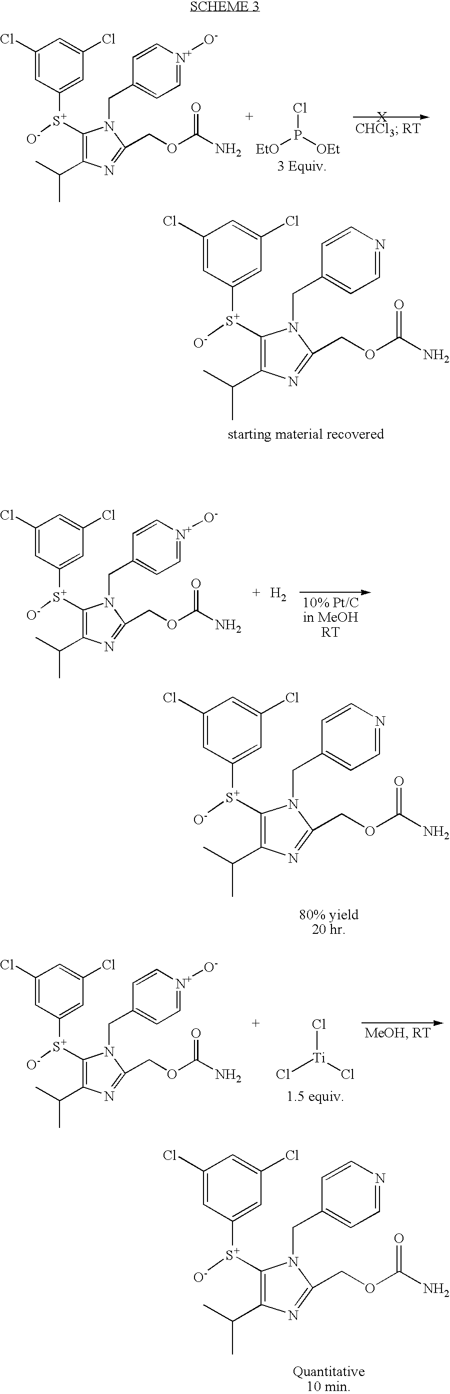

The use of platinum on carbon was effective for the hydrogenolysis reaction. However, titanium trichloride performed the reaction much faster, and it was selected as the preferable reagent to test for the reduction of C12 and C14.

(d) Preparation of M4 and M5 from dioxygenated precursors C12 and C14 (SCHEME 4): Reduction of pure compounds C12 and C14 in the presence of TiCl₃ solution (1.5 eq from a 15% TiCl₃ stock solution in aqueous HCl) in MeOH was completed in 10 min., according to general SCHEME 4, below.

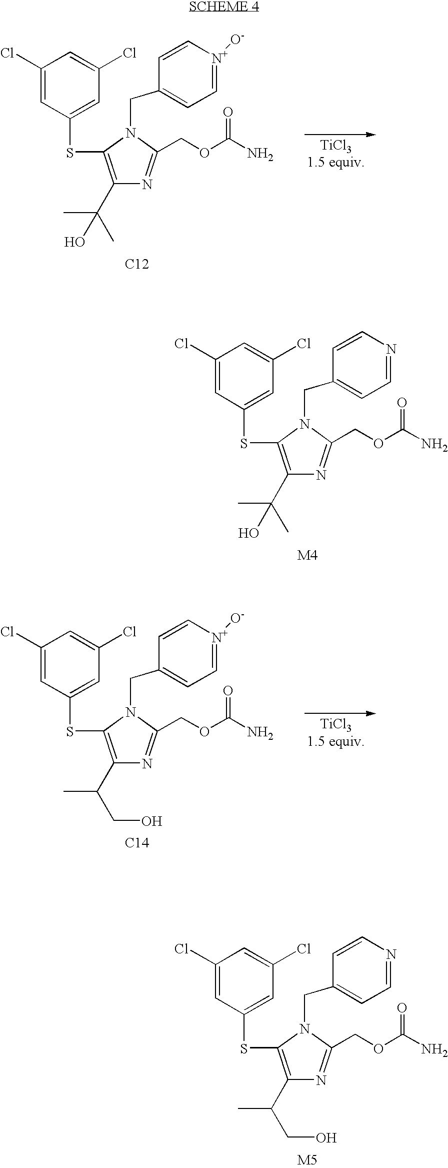

The reaction was quenched with 1 volume of 100 mM Phosphate buffer pH 8.0, stirred at room temperature for 10 min. and centrifuged at 5,000 RPM for 10 min. The supernatant was concentrated to remove methanol and then extracted (5 times) with 1 volume of chloroform to afford pure M4 and M5 after evaporation of the organic solvent. Almost quantitative recovery was observed in most runs (see procedure below for the production of M5).

3. Whole cell biotransformations for the Preparation of M4 and M5

Once reproducible and efficient cell strains were identified, 1 L reactions were run using shake flasks as culture vessels. The procedures presented below were validated at the 10 L scale.

(a) Whole cell biotransformation using Streptomyces griseus ATCC 13273

Streptomyces griseus was grown from an agar plate into a 100 ml preculture using the screening medium containing glycerol as carbon source. After 2 days culture, 10 ml of the preculture was inoculated into a 1 L culture containing fresh culture media (2% glucose as carbon source) on a 4 L shake flasks. The culture was grown for 24 hr. and substrate was added in two portions (0.2 g after 24 hr. and 0.3 g after 48 hr.). Oxidation was followed by HPLC, monitoring the amount of metabolite C12 and C14 (until approximately 10% conversion each). The cells were removed from the culture by centrifugation at 10,000 RPM and the oxidation products extracted 3 times with one volume of chloroform each. After removal of CHCl₃ in vacuo, crude product (550 mg) was obtained. The crude product was purified by preparative HPLC chromatography using the same conditions described in the analytical method (see section 2(b) above). Fifty-four mg of C12 and 60 mg of C14 were recovered. The pure deoxygenated products were then treated with TrCl3 to afford pure M4 (35 mg) and M5 (40 mg), respectively.

(b) Whole cell biotransformation using Syncephalastrum racemosum ATCC 18192

Syncephalastrum racemosum was grown from an agar plate into a 100 ml preculture using the screening medium and conditions. After 2 days, 10 ml of the preculture was inoculated into 1 L culture on a 4 L shake flask. The culture was grown for 24 hr. and substrate was added (0.2 g/L substrate load). Oxidation was followed by reverse phase HPLC and the reaction stopped after the concentration of metabolite M4 has reached approximately 20% conversion. The mycelium was removed from the culture by filtration and the oxidation products extracted 3 times with one volume of chloroform each. After removal of CHCl₃ in vacuo, crude product (150 mg) was obtained. The crude product was purified by silica gel flash chromatography, using CH₂Cl₂/Acetone/MeOH (40:1:1 and 10:1:1) as eluent, to afford 25 mg of pure M4 as the only hydroxylated product based on TLC, HPLC/MS and NMR analysis.

(c) Structural characterization of M4 and M5

¹H-NMR spectra were recorded on a Bruker DPX-300 using a QNP probe operating at 300 MHz and ¹³C-NMR spectra were recorded operating at 75 MHz. Spectra were obtained as CDCl₃ solutions (reported in ppm), using chloroform as the reference standard (7.27 ppm and 77.00 ppm) unless otherwise noted. Where peak multiplicities are reported, the following abbreviations are used: s (singlet), d (doublet), t (triplet), q (quartet), m (multiplet), br (broadened multiplet), bs (broadened singlet), dd (doublet of doublets), dt (doublet of triplets). Coupling constants, when given, are reported in Hertz (Hz).

M4: ESI: [M+1]⁺ 467.0726; calc. for C₂₀H₂₁Cl₂N₄O₃S 467.0711, ¹H NMR (CDCl₃) δ8.24 (br.d, 2H), 7.07 (br.t, 1H), 6.80 (d, 2H), 6.72 (d, 2H), 5.25 (s, 1H), 5.18 (s, 1H), 1.62 (s, 6H); ¹³C NMR (CDCl₃) δ156.12, 154.72, 148.99, 144.14, 138.45, 134.87, 127.63, 125.64, 122.85, 120.10, 110.97, 69.43, 57.44, 45.72, 29.55.

M5: M5: ESI: [M+1]⁺ 467.0726; calc. for C₂₀H₂₁Cl₂N₄O₃S 467.0711; ¹H NMR (CDCl₃) δ8.49 (br.d, 2H), 7.40 (br.d, 2H), 7.09 (t, 1H), 6.90 (t, 2H), 5.59 (s, 2H), 5.15 (s, 2H), 1.70 (m, 2H), 3.40 (m, 1H), 1.15 (d, 3H) ¹³C NMR (CDCl₃) δ 156.12, δ 148.89, 139.59, 136.03, 127.78, 126.45, 122.85, 120.10, 115.05, 67.52, 59.14, 48.48, 36.72, 17.56.

TABLE 2 provides a comparison of the antiviral activity and cytotoxicity data for CPV and the M4 and M5 metabolites.

TABLE 2
Antiviral activity and cytotoxicity of CPV and CPV metabolites^{a}

| Com- | EC₅₀ | EC₉₀ | CC₅₀ | | |
|---|---|---|---|---|---|
| pound | (uM) | (uM) | (uM) | TI^{b} | Activity |
| CPV | 0.0015 | 0.0032 | 69 | 45,667 | + |
| M4 | 0.048 | 0.11 | >320 | >6,737 | + |
| M5 | 0.047 | 0.11 | >320 | >6,882 | + |

^{a}Antiviral activity and cytotoxicity were determined measuring XTT dye reduction. Results for M4 and M5 represent the mean of two to four experiments. Results for CPV represent the mean of 9 experiments.
^{b}Therapeutic index = CC₅₀/EC_{50.}

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1. A method for preparing a metabolite of 2-carbamoyloxymethyl-5-(3,5-dichlorophenyl)thio-4-isopropyl-1-(4-pyridyl)methyl-1H-imidazole comprising:
(a) providing a cell strain selected from the group consisting of:
Streptomyces griseus, Streptomyces griseolus, Syncephalastrum racemosum, Actinoplanes sp., Streptomyces rimosus, Absidia pseudocylindrospora, Mortierella isabellina and Verticillium theobromae;
(b) reacting said cell strain with 2-carbamoyloxymethyl-5-(3,5-dichlorophenyl)thio-4-isopropyl-1-(4-pyridyl)methyl-1H-imidazole; and
(c) collecting said metabolite.

2. The method of claim 1 wherein said metabolite is an oxidative metabolite.

3. The method of claim 1 wherein said metabolite is

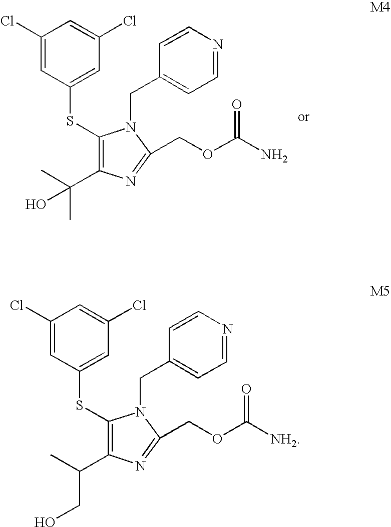

4. The method of claim 1 wherein said cell strain is Streptomyces griseus, Streptomyces griseolus or Syncephalastrum racemosum.

5. A method for preparing a metabolite of 2-carbamoyloxymethyl-5-(3,5-dichlorophenyl)thio-4-isopropyl-1-(4-pyridyl)methyl-1H-imidazole comprising:
(a) providing a bacteria cell strain selected from the group consisting of:
Actinoplanes sp., Streptomyces griseolus, Streptomyces griseus, and Streptomyces rimosus;
(b) reacting said bacteria cell strain with 2-carbamoyloxymethyl-5-(3,5-dichlorophenyl)thio-4-isopropyl-1-(4-pyridyl)methyl-1H-imidazole;
(c) producing a compound of formula 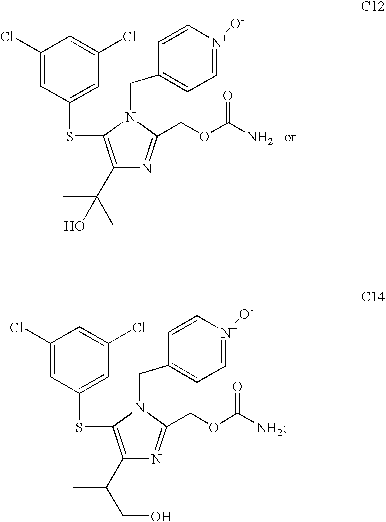
(d) reacting said compound from step (c) with TiCl₃; and
(e) collecting said metabolite.

6. The method of claim 5 wherein said metabolite is

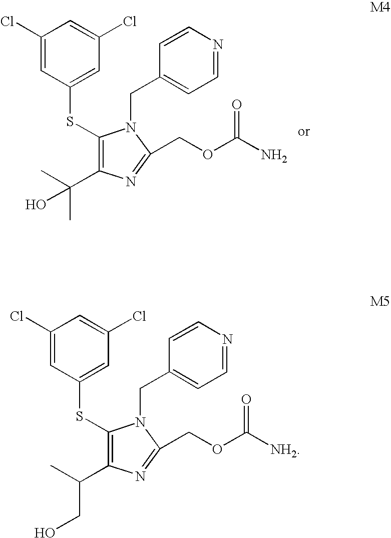

7. The method of claim 5 wherein said bacteria cell strain is Streptomyces griseus or Streptomyces griseolus.

8. A method for preparing a metabolite of 2-carbamoyloxymethyl-5-(3,5-dichlorophenyl)thio-4-isopropyl-1-(4-pyridyl)methyl-1H-imidazole comprising:
(a) providing a fungus cell strain selected from the group consisting of:
Syncephalastrum racemosum, Absidia pseudocylindrospora, Mortierella isabellina and Verticillium theobromae;
(b) reacting said cell strain with 2-carbamoyloxymethyl-5-(3,5-dichlorophenyl)thio-4-isopropyl-1-(4-pyridyl)methyl-1H-imidazole;
(c) producing a compound of formula 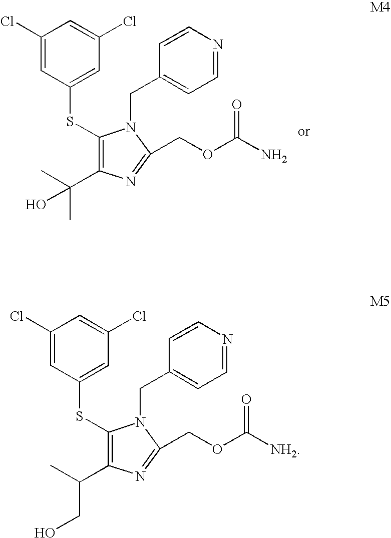
(d) reacting said compound from step (c) with TiCl₃; and
(e) collecting said metabolite.

9. The method of claim 8 wherein said metabolite is

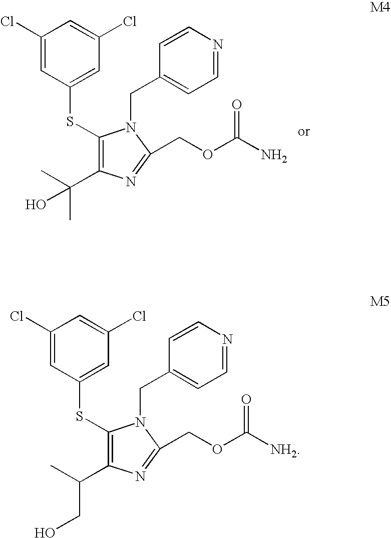

10. The method of claim 8 wherein said fungus cell strain is Syncephalastrum racemosum.
